(19) United States
(12) Patent Application Publication
(10) Pub. No.: US 2025/0099948 A1
(43) Pub. Date: Mar. 27, 2025

(54) CATALYST FOR SYNTHESIZING CYCLIC CARBONATE AND SYNTHESIS METHOD FOR CYCLIC CARBONATE

(71) Applicant: SHENZHEN CAPCHEM TECHNOLOGY CO., LTD., SHENZHEN (CN)

(72) Inventors: XUEJUN CHEN, SHENZHEN (CN); CHAOWEI CAO, SHENZHEN (CN); BANGYING WANG, SHENZHEN (CN); KAI TU, SHENZHEN (CN)

(21) Appl. No.: 18/937,044

(22) Filed: Nov. 5, 2024

Related U.S. Application Data
(63) Continuation of application No. PCT/CN2023/091510, filed on Apr. 28, 2023.

(30) Foreign Application Priority Data
Jun. 27, 2022 (CN) 202210743750.2

(51) Int. Cl. B01J 31/02; C07D 317/34

(52) U.S. Cl. CPC B01J 31/0271; C07D 317/34

(57) ABSTRACT

The present disclosure relates to a catalyst for synthesizing a cyclic carbonate and a synthesis method for a cyclic carbonate. The present disclosure provides a novel catalyst of a hydroxy quaternary phosphonium salt structure, wherein a specific type of substituent is selected, such that the catalytic effect of the catalyst is significantly improved, and meanwhile, the catalyst has superior stability. For the cyclic carbonate synthesized by using the catalyst of the present disclosure, the selectivity of the product is as high as 99.8%, and the yield is as high as 99%; the catalyst can still maintain a relatively high yield and relatively good stability after being repeatedly used.

### CROSS REFERENCE TO RELATED APPLICATIONS

This application is a continuation application of International Application No. PCT/CN2023/091510, filed on Apr. 28, 2023, which claims priority to Chinese Patent Application No. 202210743750.2, filed on Jun. 27, 2022. The disclosures of the aforementioned patent applications are hereby incorporated by reference in their entireties.

### TECHNICAL FIELD

The present disclosure relates to the technical field of catalysis, and particularly relates to a catalyst for synthesizing a cyclic carbonate and a synthesis method for a cyclic carbonate.

### BACKGROUND

In recent years, the influence of carbon dioxide on global warming has been increasing with the burning of fossil fuels, and the conversion of carbon dioxide as a Cl resource to high value-added chemicals is an effective means to alleviate the energy crisis and environmental problems. The carbonylation reaction of carbon dioxide with an epoxy compound to synthesize a cyclic carbonate is relatively representative. In recent years, cyclic carbonates have been widely used as high value-added chemicals in the fields of fine chemical engineering, lithium battery manufacturing, and the synthesis of polycarbonates and polyurethanes. The preparation methods for cyclic carbonates mainly include a phosgene method, an ester exchange method, and a cycloaddition method of carbon dioxide with an epoxy compound. Moreover, the cycloaddition of carbon dioxide with an epoxy compound to prepare a cyclic carbonate is a green chemical method with 100% atom economy, which has been receiving much attention from academic and industrial fields.

Under natural conditions, the carbon dioxide and the epoxy compound will be difficult to react, or the efficiency of the reaction of the two to form a cyclic carbonate is relatively low. Therefore, selecting a proper catalyst can effectively improve the efficiency of the reaction of the carbon dioxide with the epoxy compound to generate a cyclic carbonate. Most of the reported production processes of cyclic carbonates use a binary catalyst consisting of a Lewis acid metal and a Lewis base, among which the used Lewis acid metal includes: an alkali metal halide, an alkaline earth metal halide, a transition metal salt, a transition metal complex, or a tetradentate Schiff base metal complex; the used Lewis base includes an organic base, a quaternry ammonium salt, an imidazolium salt, a solid base (e.g., metal oxide), a crown ether, a molecular sieve, and the like. These catalyst systems more or less have the problems of low catalytic activity, poor stability, harsh reaction conditions, use of organic solvents with strong toxicity, high catalyst cost, and the like. Therefore, the development of a catalyst with mild reaction conditions, good catalytic performance, and low catalytic cost is an urgent technical problem to be solved in the field of cyclic carbonate synthesis.

### SUMMARY

Aiming at the above technical problems, the present disclosure provides a brand-new catalyst for synthesizing a cyclic carbonate, which has the advantages of good stability, low catalytic cost, high reaction efficiency, and high selectivity.

The technical solutions adopted by the present disclosure for solving the technical problems described above are as follows:

The present disclosure provides a catalyst for synthesizing a cyclic carbonate, the catalyst being a compound represented by a structural formula described below:

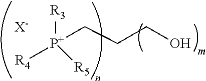

wherein R₃, R₄, and R₅ are each independently selected from a hydrogen atom and C₁-C₁₆ alkyl; X is a halogen atom; when n=1, m=2 or 3; when n=2, m=2; when n=3, m=1.

Further, R₃, R₄, and R₅ are each independently selected from C₁-C₄ alkyl.

Further, X is selected from one of Cl, Br, and I; preferably, X is selected from one of Cl and Br.

Further, the catalyst is selected from one of compounds represented by structural formulas described below:

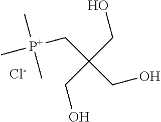

1A: [3-hydroxy-2,2-
bis(hydroxymethyl)-
propyl]trimethyl
phosphonium chloride

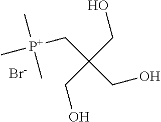

1B: [3-hydroxy-2,2-
bis(hydroxymethyl)-
propyl]trimethyl
phosphonium bromide

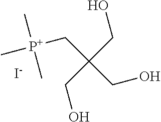

1C: [3-hydroxy-2,2-
bis(hydroxymethyl)-
propyl]trimethyl
phosphonium iodide

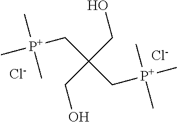

2A: [3-hydroxy-2-(hydroxy-
methyl)-2-[(trimethylphos-
phonium)methyl]propyl]
trimethylphosphonium
dichloride

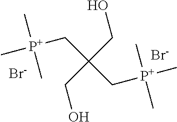

2B: [3-hydroxy-2-(hydroxy-
methyl)-2-[(trimethylphos-
phonium)methyl]propyl]
trimethylphosphonium
dibromide

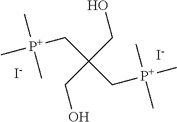

2C: [3-hydroxy-2-(hydroxy-
methyl)-2-[(trimethylphos-
phonium)methyl]propyl]
trimethylphosphonium
diiodide

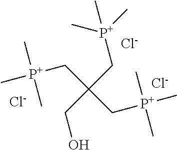

3A: [2-(hydroxymethyl)-3-
(trimethylphosphonium)-2-
[(trimethylphosphonium)-
methyl]propyl]trimethyl-
phosphonium trichloride

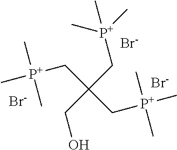

3B: [2-(hydroxymethyl)-3-
(trimethylphosphonium)-2-
[(trimethylphosphonium)-
methyl]propyl]trimethyl-
phosphonium tribromide

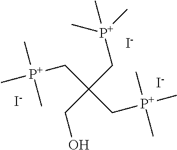

3C: [2-(hydroxymethyl)-3-
(trimethylphosphonium)-2-
[(trimethylphosphonium)-
methyl]propyl]trimethyl-
phosphonium triiodide

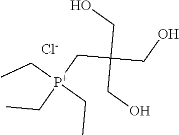

4A: [3-hydroxy-2,2-bis-
(hydroxymethyl)propyl]
triethylphosphonium
chloride

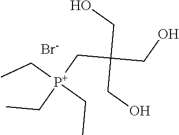

4B: [3-hydroxy-2,2-bis-
(hydroxymethyl)propyl]
triethylphosphonium
bromide

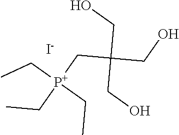

4C: [3-hydroxy-2,2-bis-
(hydroxymethyl)propyl]
triethylphosphonium
iodide

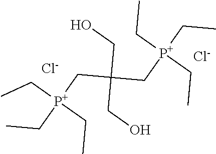 [SMILES: CC[P+](CC)(CC)CC(CO)(CO)C[P+](CC)(CC)CC ; [Cl-] ; [Cl-]]

5A: [3-hydroxy-2-(hydroxymeth-
yl)-2-[(triethylphosphonium)meth-
yl]propyl]triethylphosphonium
dichloride

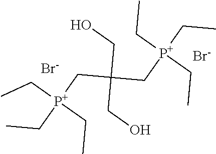 [SMILES: CC[P+](CC)(CC)CC(CO)(CO)C[P+](CC)(CC)CC ; [Br-] ; [Br-]]

5B: [3-hydroxy-2-(hydroxymethyl)-
2-[(triethylphosphonium)meth-
yl]propyl]triethylphosphonium
dibromide

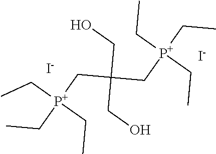 [SMILES: CC[P+](CC)(CC)CC(CO)(CO)C[P+](CC)(CC)CC ; [I-] ; [I-]]

5C: [3-hydroxy-2-(hydroxymethyl)-
2-[(triethylphosphonium)meth-
yl]propyl]triethylphosphonium
diiodide

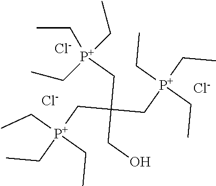 [SMILES: CC[P+](CC)(CC)CC(CO)(C[P+](CC)(CC)CC)C[P+](CC)(CC)CC ; [Cl-] ; [Cl-] ; [Cl-]]

6A: [2-(hydroxymethyl)-3-triethyl-
(phosphonium)-2-[(triethylphos-
phonium)methyl]propyl]triethyl-
phosphonium trichloride

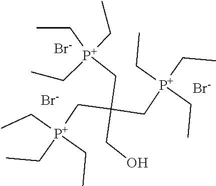 [SMILES: CC[P+](CC)(CC)CC(CO)(C[P+](CC)(CC)CC)C[P+](CC)(CC)CC ; [Br-] ; [Br-] ; [Br-]]

6B: [2-(hydroxymethyl)-3-(triethyl-
phosphonium)-2-[(triethylphos-
phonium)methyl]propyl]triethyl-
phosphonium tribromide

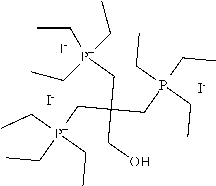 [SMILES: CC[P+](CC)(CC)CC(CO)(C[P+](CC)(CC)CC)C[P+](CC)(CC)CC ; [I-] ; [I-] ; [I-]]

6C: [2-(hydroxymethyl)-3-(triethyl-
phosphonium)-2-[(triethylphos-
phonium)methyl]propyl]triethyl-
phosphonium triiodide

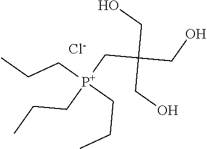 [SMILES: CCC[P+](CCC)(CCC)CC(CO)(CO)CO ; [Cl-]]

7A: [3-hydroxy-2,2-bis(hydroxy-
methyl)propyl]tripropylphos-
phonium chloride

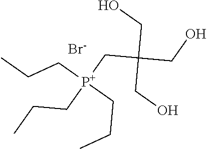 [SMILES: CCC[P+](CCC)(CCC)CC(CO)(CO)CO ; [Br-]]

7B: [3-hydroxy-2,2-bis(hydroxy-
methyl)propyl]tripropylphos-
phonium bromide

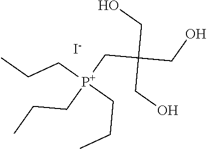 [SMILES: CCC[P+](CCC)(CCC)CC(CO)(CO)CO ; [I-]]

7C: [3-hydroxy-2,2-bis(hydroxy-
methyl)propyl]tripropylphos-
phonium iodide

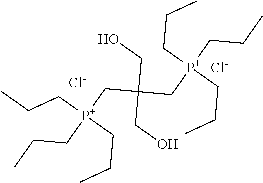 [SMILES: CCC[P+](CCC)(CCC)CC(CO)(CO)C[P+](CCC)(CCC)CCC ; [Cl-] ; [Cl-]]

8A: [3-hydroxy-2-(hydroxymethyl)-2-
[(triethylphosphonium)methyl]propyl]
tripropylphosphonium dichloride

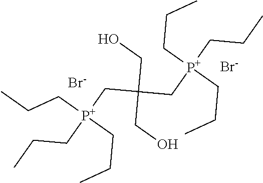 [SMILES: CCC[P+](CCC)(CCC)CC(CO)(CO)C[P+](CCC)(CCC)CCC ; [Br-] ; [Br-]]

8B: [3-hydroxy-2-(hydroxymethyl)-2-
[(triethylphosphonium)methyl]propyl]
tripropylphosphonium dibromide

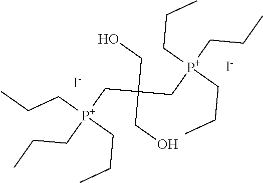 [SMILES: CCC[P+](CCC)(CCC)CC(CO)(CO)C[P+](CCC)(CCC)CCC ; [I-] ; [I-]]

8C: [3-hydroxy-2-(hydroxymethyl)-2-
[(tripropylphosphonium)methyl]propyl]
tripropylphosphonium diiodide

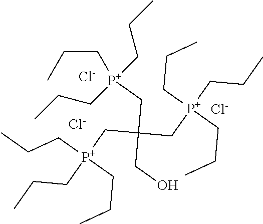 [SMILES: CCC[P+](CCC)(CCC)CC(CO)(C[P+](CCC)(CCC)CCC)C[P+](CCC)(CCC)CCC ; [Cl-] ; [Cl-] ; [Cl-]]

9A: [2-(hydroxymethyl)-3-(tripropyl-
phosphonium)-2-[(tripropylphos-
phonium)methyl]propyl]tripropyl-
phosphonium trichloride

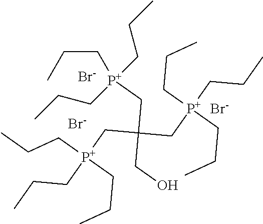 [SMILES: CCC[P+](CCC)(CCC)CC(CO)(C[P+](CCC)(CCC)CCC)C[P+](CCC)(CCC)CCC ; [Br-] ; [Br-] ; [Br-]]

9B: [2-(hydroxymethyl)-3-(tripropyl-
phosphonium)-2-[(tripropylphos-
phonium)methyl]propyl]tripropyl-
phosphonium tribromide

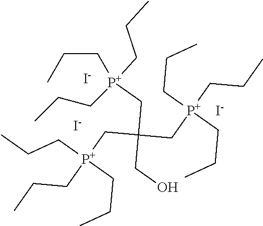 [SMILES: CCC[P+](CCC)(CCC)CC(CO)(C[P+](CCC)(CCC)CCC)C[P+](CCC)(CCC)CCC ; [I-] ; [I-] ; [I-]]

9C: [2-(hydroxymethyl)-3-(tripropyl-
phosphonium)-2-[(tripropylphos-
phonium)methyl]propyl]tripropyl-
phosphonium triiodide

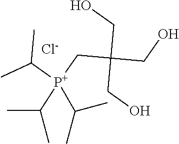 [SMILES: CC(C)[P+](CC(CO)(CO)CO)(C(C)C)C(C)C ; [Cl-]]

10A: [3-hydroxy-2,2-bis-
(hydroxymethyl)propyl]
tripropylphosphonium
chloride

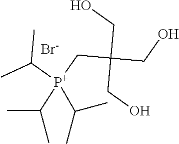 [SMILES: CC(C)[P+](CC(CO)(CO)CO)(C(C)C)C(C)C ; [Br-]]

10B: [3-hydroxy-2,2-bis-
(hydroxymethyl)propyl]
triisopropylphosphonium
bromide

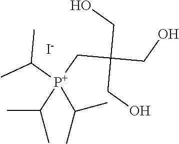 [SMILES: CC(C)[P+](CC(CO)(CO)CO)(C(C)C)C(C)C ; [I-]]

10C: [3-hydroxy-2,2-bis-
(hydroxymethyl)propyl]
triisopropylphosphonium
iodide

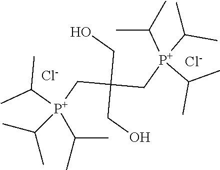 [SMILES: CC(C)[P+](CC(CO)(CO)C[P+](C(C)C)(C(C)C)C(C)C)(C(C)C)C(C)C ; [Cl-] ; [Cl-]]

11A: [3-hydroxy-2-(hydroxymeth-
yl)-2-[(triisopropylphosphonium)-
methyl]propyl]triisopropylphos-
phonium dichloride

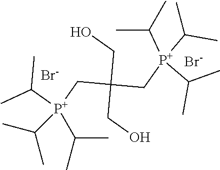 [SMILES: CC(C)[P+](CC(CO)(CO)C[P+](C(C)C)(C(C)C)C(C)C)(C(C)C)C(C)C ; [Br-] ; [Br-]]

11B: [3-hydroxy-2-(hydroxymethyl)-
2-[(triisopropylphosphonium)-
methyl]propyl]triisopropylphos-
phonium dibromide

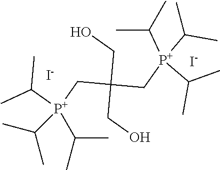 [SMILES: CC(C)[P+](CC(CO)(CO)C[P+](C(C)C)(C(C)C)C(C)C)(C(C)C)C(C)C ; [I-] ; [I-]]

11C: [3-hydroxy-2-(hydroxymeth
yl)-2-[(triisopropylphosphon-
ium)methyl]propyl]triisopropyl-
phosphonium diiodide

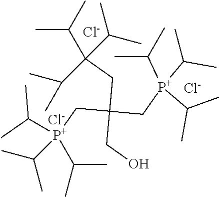 [SMILES: CC(C)C(CC(CO)(C[P+](C(C)C)(C(C)C)C(C)C)C[P+](C(C)C)(C(C)C)C(C)C)(C(C)C)C(C)C ; [Cl-] ; [Cl-] ; [Cl-]]

12A: [2-(hydroxymethyl)-3-(tri-
isopropylphosphonium)-2-[(tri-
isopropylphosphonium)methyl]
propyl]triisopropylphosphonium
chloride

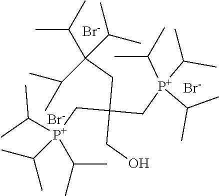 [SMILES: CC(C)C(CC(CO)(C[P+](C(C)C)(C(C)C)C(C)C)C[P+](C(C)C)(C(C)C)C(C)C)(C(C)C)C(C)C ; [Br-] ; [Br-] ; [Br-]]

12B: [2-(hydroxymethyl)-3-(tri-
isopropylphosphonium)-2-[(tri-
isopropylphosphonium)methyl]-
propyl]triisopropylphosphonium
tribromide

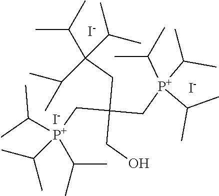 [SMILES: CC(C)C(CC(CO)(C[P+](C(C)C)(C(C)C)C(C)C)C[P+](C(C)C)(C(C)C)C(C)C)(C(C)C)C(C)C ; [I-] ; [I-] ; [I-]]

12C: [2-(hydroxymethyl)-3-(tri-
isopropylphosphonium)-2-[(tri-
isopropylphosphonium)methyl]
propyl]triisopropylphosphonium
triiodide

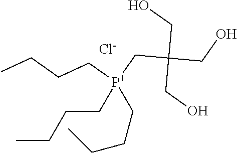 [SMILES: CCCC[P+](CCCC)(CCCC)CC(CO)(CO)CO ; [Cl-]]

13A: [3-hydroxy-2,2-bis(hydroxy-
methyl)propyl]tributylphospho-
nium chloride

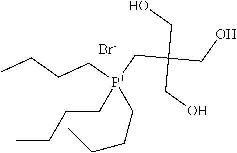 [SMILES: CCCC[P+](CCCC)(CCCC)CC(CO)(CO)CO ; [Br-]]

13B: [3-hydroxy-2,2-bis(hydroxy-
methyl)propyl]tributylphospho-
nium bromide

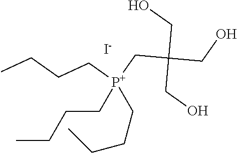 [SMILES: CCCC[P+](CCCC)(CCCC)CC(CO)(CO)CO ; [I-]]

13C: [3-hydroxy-2,2-bis(hydroxy-
methyl)propyl]tributylphospho-
nium iodide

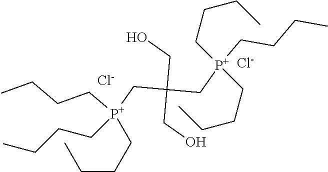 [SMILES: CCCC[P+](CCCC)(CCCC)CC(CO)(CO)C[P+](CCCC)(CCCC)CCCC ; [Cl-] ; [Cl-]]

14A: [3-hydroxy-2-(hydroxymethyl)-2-[(tributyl-
phosphonium)methyl]propyl]tributylphospho-
nium dichloride

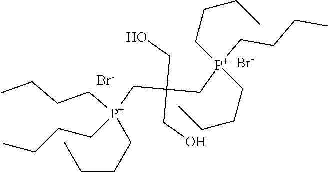 [SMILES: CCCC[P+](CCCC)(CCCC)CC(CO)(CO)C[P+](CCCC)(CCCC)CCCC ; [Br-] ; [Br-]]

14B: [3-hydroxy-2-(hydroxymethyl)-2-[(tributyl-
phosphonium)methyl]propyl]tributylphosphonium
dibromide

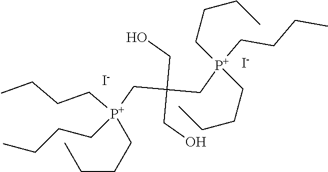 [SMILES: CCCC[P+](CCCC)(CCCC)CC(CO)(CO)C[P+](CCCC)(CCCC)CCCC ; [I-] ; [I-]]

14C: [3-hydroxy-2-(hydroxymethyl)-2-[(tributyl-
phosphonium)methyl]propyl]tributylphosphonium
diiodide

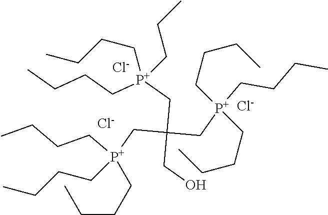 [SMILES: CCCC[P+](CCC)(CCCC)CC(CO)(C[P+](CCCC)(CCCC)CCCC)C[P+](CCCC)(CCCC)CCCC ; [Cl-] ; [Cl-] ; [Cl-]]

15A: [2-(hydroxymethyl)-3-(tributylphosphonium)-
2-[(tributylphosphonium)methyl]propyl]tributyl-
phosphonium trichloride

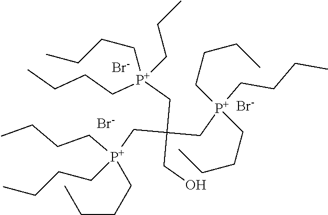 [SMILES: CCCC[P+](CCC)(CCCC)CC(CO)(C[P+](CCCC)(CCCC)CCCC)C[P+](CCCC)(CCCC)CCCC ; [Br-] ; [Br-] ; [Br-]]

15B: [2-(hydroxymethyl)-3-(tributylphosphonium)-
2-[(tributylphosphonium)methyl]propyl]tributyl-
phosphonium tribromide

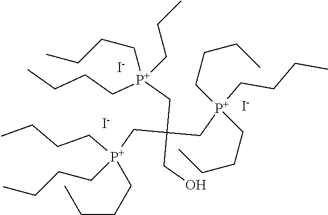 [SMILES: CCCC[P+](CCC)(CCCC)CC(CO)(C[P+](CCCC)(CCCC)CCCC)C[P+](CCCC)(CCCC)CCCC ; [I-] ; [I-] ; [I-]]

15C: [2-(hydroxymethyl)-3-(tributylphosphonium)-
2-[(tributylphosphonium)methyl]propyl]tributyl-
phosphonium triiodide

In another aspect, the present disclosure further provides a synthesis method for a cyclic carbonate, in which carbon dioxide and an epoxy compound are taken as raw materials and reacted under the action of the catalyst to synthesize the cyclic carbonate.

Further, a structural formula of the epoxy compound is:

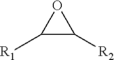

wherein when R₁=H, R₂ is one of H, CH₃, C₂H₅, CH₂Cl, C₂H₃, C₄H₉O, C₄H₉, C₆H₅, and C₇H₇O; when R₁≠H, the epoxy compound is cyclohexene oxide.

Specifically, a structural formula of the epoxy compound is:

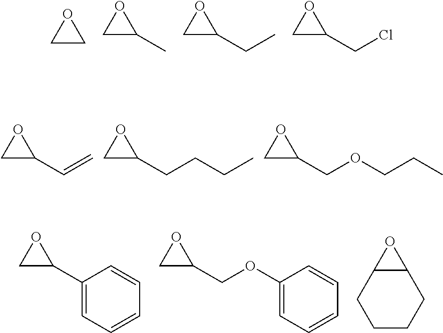

Further, a molar ratio of the hydroxyazocyclic quaternary ammonium salt to the epoxy compound is 1×10⁻³-2.5×10⁻³:1.

Further, a pressure of the reaction is 0.1-10 MPa.

Further, a temperature of the reaction is 40-220° C.

Further, a time of the reaction is 0.5-6 h.

The present disclosure has the following beneficial effects:

(1) The present disclosure provides a novel catalyst for synthesizing a cyclic carbonate, the catalyst being of a hydroxy quaternary phosphonium salt structure, wherein by selecting a specific type of substituent for the hydroxy quaternary phosphonium salt, the catalytic effect of the catalyst is significantly improved, and the catalyst has superior stability. The selectivity of the cyclic carbonate synthesized by using the catalyst of the present disclosure can be as high as 99.8%, and the yield can be as high as 99%; the catalyst can still remain a relatively high cyclic carbonate yield after be repeatedly used for above 3 times, and the catalyst has good stability.

(2) According to the synthetic method for a cyclic carbonate of the present disclosure, the cyclic carbonate can be efficiently synthesized under mild reaction conditions by using the catalyst of the hydroxy quaternary phosphonium salt structure, and the catalyst has low cost, high selectivity, and good thermal stability, and can be repeatedly used for many times.

### BRIEF DESCRIPTION OF THE DRAWINGS

In order to more clearly illustrate the technical solutions in the embodiments of the present invention or in the prior art, the drawings required to be used in the description of the embodiments or the prior art are briefly introduced below. It is obvious that the drawings in the description below are some embodiments recorded in the present invention, and those of ordinary skill in the art can obtain other drawings according to the drawings provided herein without creative efforts.

FIG. 1 shows the results of gas chromatographic analysis on the product obtained in Example 1 of the present disclosure;

FIG. 2 shows the results of gas chromatographic analysis on the product obtained in Example 2 of the present disclosure.

### DETAILED DESCRIPTION

The technical solutions of the present disclosure will be clearly and completely described below in conjunction with specific examples, and it is obvious that the described examples are only a part of the examples of the present disclosure but not all of them. Based on the examples of the present disclosure, all other examples obtained by those of ordinary skill in the art without creative effort shall fall within the protection scope of the present disclosure.

The catalyst used for synthesizing a cyclic carbonate of the present disclosure is selected from compounds represented by the following structural formula:

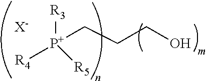

wherein R₃, R₄, and R₅ are each independently selected from a hydrogen atom and C₁-C₁₆ alkyl; X is a halogen atom; when n=1, m=2 or 3; when n=2, m=2; when n=3, m=1.

Specifically, the synthesis method for the catalyst, taking the synthesis of

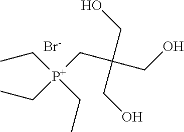

as an example, comprises the following steps:

A mixture of 7.5 g of triethylphosphine (63.5 mmol) and 10 g of 2-(bromomethyl)-2-(hydroxymethyl)propane-1,3-diol (0.5 mmol) is heated at 130° C. for 8 h under magnetic stirring. After cooling to room temperature, the obtained solid is washed 3 times with acetonitrile, and the residue is dried in an oven at 100° C. for 2 h to obtain 15.58 g of [3-hydroxy-2,2-bis(hydroxymethyl)propyl]triethylphosphonium bromide as a white powder (yield: 98%).

In the method for synthesizing a cyclic carbonate of the present disclosure, carbon dioxide and an epoxy compound are taken as reaction raw materials, and a reaction general formula thereof is:

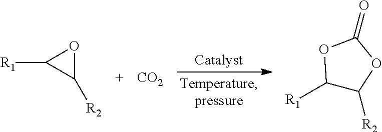

wherein R₁ and R₂ are substituents on a ring of the epoxy compound, and when R₁=H, R₂ is one of H (ethylene oxide), CH₃ (propylene oxide), C₂H₅ (butylene oxide), CH₂Cl (epichlorohydrin), C₂H₃ (epoxybutene), C₄H₉O (2-propoxymethylethylene oxide), C₄H₉ (epoxyhexane), C₆H₅ (epoxyphenylethane), or C₇H₇O (2-(phenoxymethyl)ethylene oxide); when R₁≠H, the epoxy compound is cyclohexene oxide.

Specifically, a structural formula of the epoxy compound is:

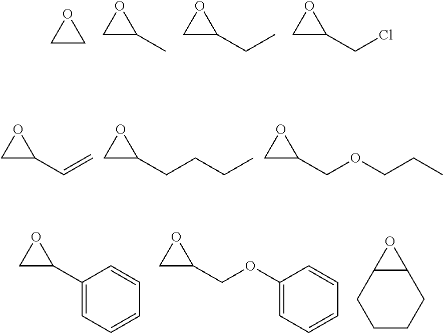

A molar ratio of the catalyst to the epoxy compound is 1×10⁻³-2.5×10⁻³:1, and the cyclic carbonate is synthesized under the conditions that a reaction pressure is 0.1-10 MPa, a temperature is 40-220° C., and a reaction time is 0.5-6 h. The method has mild reaction conditions, and the used catalyst has the advantages of low cost, high selectivity, good thermal stability, and the ability to be repeatedly used for many times.

The method for synthesizing a cyclic carbonate of the present disclosure will be further described below in conjunction with specific examples.

### Example 1

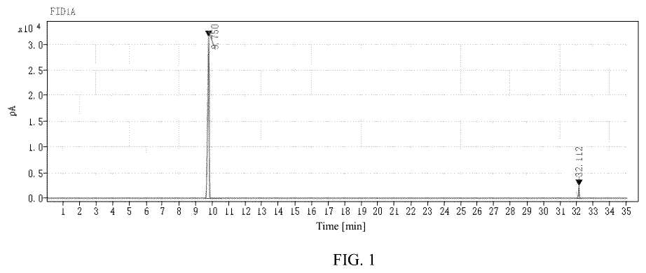

1 mol ethylene oxide and 1.6 mmol [3-hydroxy-2,2-bis(hydroxymethyl)propyl]triethylphosphonium bromide were sequentially added into a 25 mL stainless steel high-pressure reactor with a polytetrafluoroethylene lining, the reactor was sealed, carbon dioxide at a proper pressure was charged, the reactor was slowly heated to 120° C., then the pressure of the carbon dioxide was controlled to be 3 MPa, and the reaction was performed for 0.5 h. The reactor was cooled to room temperature and depressurized, the carbon dioxide was absorbed by a saturated sodium bicarbonate solution, and the obtained liquid was distilled under reduced pressure to obtain a product. The peak time of the product was consistent with that of a standard sample through gas chromatographic analysis (FIG. 1), indicating that the product was ethylene carbonate, the selectivity of the product was 99.8%, and the yield was 99%.

The distillation residue was transferred to the high-pressure reactor and used as a catalyst for the next catalytic reaction. After the catalyst was repeatedly used for 3 times, the selectivity of a synthesized product was 99%, and the yield was 98.5%.

### Example 2

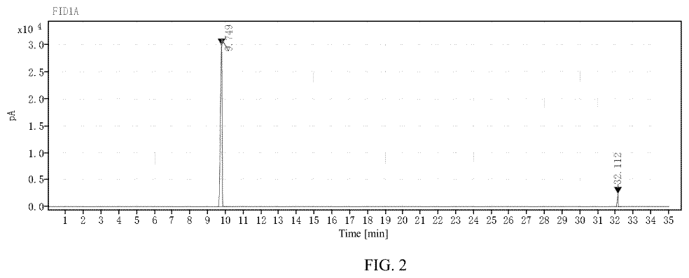

1 mol ethylene oxide and 1.4 mmol [3-hydroxy-2-(hydroxymethyl)-2-[(trimethylphosphium)methyl]propyl]trimethylphosphonium dichloride were sequentially added into a 25 mL stainless steel high-pressure reactor with a polytetrafluoroethylene lining, the reactor was sealed, carbon dioxide at a proper pressure was charged, the reactor was slowly heated to 130° C., then the pressure of the carbon dioxide was controlled to be 4 MPa, and the reaction was performed for 0.6 h. The reactor was cooled to room temperature and depressurized, the carbon dioxide was absorbed by a saturated sodium bicarbonate solution, and the obtained liquid was distilled under reduced pressure to obtain a product. The peak time of the product was consistent with that of a standard sample through gas chromatographic analysis (FIG. 2), indicating that the product was ethylene carbonate, the selectivity of the product was 99.5%, and the yield was 99%.

The distillation residue was transferred to the high-pressure reactor and used as a catalyst for the next catalytic reaction. After the catalyst was repeatedly used for 3 times, the selectivity of a synthesized product was 99%, and the yield was 98.5%.

### Example 3

1 mol propylene oxide and 1.2 mmol [3-hydroxy-2,2-bis(hydroxymethyl)propyl]trimethylphosphonium iodide were sequentially added into a 25 mL stainless steel high-pressure reactor with a polytetrafluoroethylene lining, the reactor was sealed, carbon dioxide at a proper pressure was charged, the reactor was slowly heated to 140° C., then the pressure of the carbon dioxide was controlled to be 2 MPa, and the reaction was performed for 0.7 h. The reactor was cooled to room temperature and depressurized, the carbon dioxide was absorbed by a saturated sodium bicarbonate solution, and the obtained liquid was distilled under reduced pressure to obtain a product. The peak time of the product was consistent with that of a standard sample through gas chromatographic analysis, indicating that the product was ethylene carbonate, the selectivity of the product was 99%, and the yield was 98%.

The distillation residue was transferred to the high-pressure reactor and used as a catalyst for the next catalytic reaction. After the catalyst was repeatedly used for 3 times, the selectivity of a synthesized product was 97.8%, and the yield was 96.9%.

### Example 4

1 mol propylene oxide and 1.6 mmol [2-(hydroxymethyl)-3-(trimethylphosphonium)-2-[(trimethylphosphonium)methyl]propyl]trimethylphosphonium trichloride were sequentially added into a 25 mL stainless steel high-pressure reactor with a polytetrafluoroethylene lining, the reactor was sealed, carbon dioxide at a proper pressure was charged, the reactor was slowly heated to 120° C., then the pressure of the carbon dioxide was controlled to be 3 MPa, and the reaction was performed for 0.5 h. The reactor was cooled to room temperature and depressurized, the carbon dioxide was absorbed by a saturated sodium bicarbonate solution, and the obtained liquid was distilled under reduced pressure to obtain a product. The peak time of the product was consistent with that of a standard sample through gas chromatographic analysis, indicating that the product was ethylene carbonate, the selectivity of the product was 98.3%, and the yield was 96.7%.

The distillation residue was transferred to the high-pressure reactor and used as a catalyst for the next catalytic reaction. After the catalyst was repeatedly used for 3 times, the selectivity of a synthesized product was 95.9%, and the yield was 95%.

### Example 5

1 mol butylene oxide and 1.6 mmol [3-hydroxy-2-(hydroxymethyl)-2-[(triethylphosphonimn)methyl]propyl]triethylphosphonium dibromide were sequentially added into a 25 mL stainless steel high-pressure reactor with a polytetrafluoroethylene lining, the reactor was sealed, carbon dioxide at a proper pressure was charged, the reactor was slowly heated to 120° C., then the pressure of the carbon dioxide was controlled to be 3 MPa, and the reaction was performed for 0.5 h. The reactor was cooled to room temperature and depressurized, the carbon dioxide was absorbed by a saturated sodium bicarbonate solution, and the obtained liquid was distilled under reduced pressure to obtain a product. The peak time of the product was consistent with that of a standard sample through gas chromatographic analysis, indicating that the product was ethylene carbonate, the selectivity of the product was 98%, and the yield was 96%.

The distillation residue was transferred to the high-pressure reactor and used as a catalyst for the next catalytic reaction. After the catalyst was repeatedly used for 3 times, the selectivity of a synthesized product was 97%, and the yield was 95%.

### Example 6

1 mol butylene oxide and 1.6 mmol [2-(hydroxymethyl)-3-(triethylphosphonium)-2-[(triethylphosphonium)methyl]propyl]triethylphosphonium trichloride were sequentially added into a 25 mL stainless steel high-pressure reactor with a polytetrafluoroethylene lining, the reactor was sealed, carbon dioxide at a proper pressure was charged, the reactor was slowly heated to 120° C., then the pressure of the carbon dioxide was controlled to be 5 MPa, and the reaction was performed for 0.2 h. The reactor was cooled to room temperature and depressurized, the carbon dioxide was absorbed by a saturated sodium bicarbonate solution, and the obtained liquid was distilled under reduced pressure to obtain a product. The peak time of the product was consistent with that of a standard sample through gas chromatographic analysis, indicating that the product was ethylene carbonate, the selectivity of the product was 98%, and the yield was 96%.

The distillation residue was transferred to the high-pressure reactor and used as a catalyst for the next catalytic reaction. After the catalyst was repeatedly used for 3 times, the selectivity of a synthesized product was 97%, and the yield was 96%.

### Example 7

1 mol epoxybutene and 2.5 mmol [3-hydroxy-2,2-bis(hydroxymethyl)propyl]tripropylphosphonium chloride were sequentially added into a 25 mL stainless steel high-pressure reactor with a polytetrafluoroethylene lining, the reactor was sealed, carbon dioxide at a proper pressure was charged, the reactor was slowly heated to 120° C., then the pressure of the carbon dioxide was controlled to be 6 MPa, and the reaction was performed for 0.2 h. The reactor was cooled to room temperature and depressurized, the carbon dioxide was absorbed by a saturated sodium bicarbonate solution, and the obtained liquid was distilled under reduced pressure to obtain a product. The peak time of the product was consistent with that of a standard sample through gas chromatographic analysis, indicating that the product was ethylene carbonate, the selectivity of the product was 98%, and the yield was 97%.

The distillation residue was transferred to the high-pressure reactor and used as a catalyst for the next catalytic reaction. After the catalyst was repeatedly used for 3 times, the selectivity of a synthesized product was 97%, and the yield was 96.5%.

### Example 8

1 mol epoxybutene and 1.5 mmol [3-hydroxy-2-(hydroxymethyl)-2-[(tripropylphosphonium)methyl]propyl]tripropylphosphonium dibromide were sequentially added into a 25 mL stainless steel high-pressure reactor with a polytetrafluoroethylene lining, the reactor was sealed, carbon dioxide at a proper pressure was charged, the reactor was slowly heated to 130° C., then the pressure of the carbon dioxide was controlled to be 2 MPa, and the reaction was performed for 2 h. The reactor was cooled to room temperature and depressurized, the carbon dioxide was absorbed by a saturated sodium bicarbonate solution, and the obtained liquid was distilled under reduced pressure to obtain a product. The peak time of the product was consistent with that of a standard sample through gas chromatographic analysis, indicating that the product was ethylene carbonate, the selectivity of the product was 98.5%, and the yield was 98%.

The distillation residue was transferred to the high-pressure reactor and used as a catalyst for the next catalytic reaction. After the catalyst was repeatedly used for 3 times, the selectivity of a synthesized product was 98%, and the yield was 97%.

### Example 9

1 mol epichlorohydrin and 1.2 mmol [2-(hydroxymethyl)-3-(tripropylphosphonium)-2-[(tripropylphosphonium)methyl]propyl]tripropylphosphonium triiodide were sequentially added into a 25 mL stainless steel high-pressure reactor with a polytetrafluoroethylene lining, the reactor was sealed, carbon dioxide at a proper pressure was charged, the reactor was slowly heated to 130° C., then the pressure of the carbon dioxide was controlled to be 3 MPa, and the reaction was performed for 3 h. The reactor was cooled to room temperature and depressurized, the carbon dioxide was absorbed by a saturated sodium bicarbonate solution, and the obtained liquid was distilled under reduced pressure to obtain a product. The peak time of the product was consistent with that of a standard sample through gas chromatographic analysis, indicating that the product was ethylene carbonate, the selectivity of the product was 98.3%, and the yield was 97%.

The distillation residue was transferred to the high-pressure reactor and used as a catalyst for the next catalytic reaction. After the catalyst was repeatedly used for 3 times, the selectivity of a synthesized product was 97.6%, and the yield was 95%.

### Example 10

1 mol epichlorohydrin and 1.0 mmol [3-hydroxy-2,2-bis(hydroxymethyl)propyl]triisopropylphosphonium chloride were sequentially added into a 25 mL stainless steel high-pressure reactor with a polytetrafluoroethylene lining, the reactor was sealed, carbon dioxide at a proper pressure was charged, the reactor was slowly heated to 140° C., then the pressure of the carbon dioxide was controlled to be 3 MPa, and the reaction was performed for 2.5 h. The reactor was cooled to room temperature and depressurized, the carbon dioxide was absorbed by a saturated sodium bicarbonate solution, and the obtained liquid was distilled under reduced pressure to obtain a product. The peak time of the product was consistent with that of a standard sample through gas chromatographic analysis, indicating that the product was ethylene carbonate, the selectivity of the product was 98%, and the yield was 97.6%.

The distillation residue was transferred to the high-pressure reactor and used as a catalyst for the next catalytic reaction. After the catalyst was repeatedly used for 3 times, the selectivity of a synthesized product was 98%, and the yield was 97.3%.

### Example 11

1 mol 2-propoxymethylethylene oxide and 1.1 mmol [3-hydroxy-2-(hydroxymethyl)-2-[(triisopropylphosphonium)methyl]propyl]triisopropylphosphonium dibromide were sequentially added into a 25 mL stainless steel high-pressure reactor with a polytetrafluoroethylene lining, the reactor was sealed, carbon dioxide at a proper pressure was charged, the reactor was slowly heated to 120° C., then the pressure of the carbon dioxide was controlled to be 3 MPa, and the reaction was performed for 4 h. The reactor was cooled to room temperature and depressurized, the carbon dioxide was absorbed by a saturated sodium bicarbonate solution, and the obtained liquid was distilled under reduced pressure to obtain a product. The peak time of the product was consistent with that of a standard sample through gas chromatographic analysis, indicating that the product was ethylene carbonate, the selectivity of the product was 98%, and the yield was 97.3%.

The distillation residue was transferred to the high-pressure reactor and used as a catalyst for the next catalytic reaction. After the catalyst was repeatedly used for 3 times, the selectivity of a synthesized product was 96.5%, and the yield was 96%.

### Example 12

1 mol 2-propoxymethylethylene oxide and 1.2 mmol [2-(hydroxymethyl)-3-(triisopropylphosphonium)-2-[(triisopropylphosphonium)methyl]propyl]triisopropylphosphonium triiodide were sequentially added into a 25 mL stainless steel high-pressure reactor with a polytetrafluoroethylene lining, the reactor was sealed, carbon dioxide at a proper pressure was charged, the reactor was slowly heated to 130° C., then the pressure of the carbon dioxide was controlled to be 3 MPa, and the reaction was performed for 0.3 h. The reactor was cooled to room temperature and depressurized, the carbon dioxide was absorbed by a saturated sodium bicarbonate solution, and the obtained liquid was distilled under reduced pressure to obtain a product. The peak time of the product was consistent with that of a standard sample through gas chromatographic analysis, indicating that the product was ethylene carbonate, the selectivity of the product was 97.5%, and the yield was 97%.

The distillation residue was transferred to the high-pressure reactor and used as a catalyst for the next catalytic reaction. After the catalyst was repeatedly used for 3 times, the selectivity of a synthesized product was 96.5%, and the yield was 96%.

### Example 13

1 mol epoxyphenylethane and 1.6 mmol [3-hydroxy-2,2-bis(hydroxymethyl)propyl]tributylphosphonium chloride were sequentially added into a 25 mL stainless steel high-pressure reactor with a polytetrafluoroethylene lining, the reactor was sealed, carbon dioxide at a proper pressure was charged, the reactor was slowly heated to 130° C., then the pressure of the carbon dioxide was controlled to be 8 MPa, and the reaction was performed for 0.3 h. The reactor was cooled to room temperature and depressurized, the carbon dioxide was absorbed by a saturated sodium bicarbonate solution, and the obtained liquid was distilled under reduced pressure to obtain a product. The peak time of the product was consistent with that of a standard sample through gas chromatographic analysis, indicating that the product was ethylene carbonate, the selectivity of the product was 98%, and the yield was 97%.

The distillation residue was transferred to the high-pressure reactor and used as a catalyst for the next catalytic reaction. After the catalyst was repeatedly used for 3 times, the selectivity of a synthesized product was 97%, and the yield was 96.5%.

### Example 14

1 mol 2-(phenoxymethyl)ethylene oxide and 2.0 mmol [3-hydroxy-2-(hydroxymethyl)-2-[(tributylphosphonium)methyl]propyl]tributylphosphonium dibromide were sequentially added into a 25 mL stainless steel high-pressure reactor with a polytetrafluoroethylene lining, the reactor was sealed, carbon dioxide at a proper pressure was charged, the reactor was slowly heated to 120° C., then the pressure of the carbon dioxide was controlled to be 10 MPa, and the reaction was performed for 0.1 h. The reactor was cooled to room temperature and depressurized, the carbon dioxide was absorbed by a saturated sodium bicarbonate solution, and the obtained liquid was distilled under reduced pressure to obtain a product. The peak time of the product was consistent with that of a standard sample through gas chromatographic analysis, indicating that the product was ethylene carbonate, the selectivity of the product was 98%, and the yield was 97%.

The distillation residue was transferred to the high-pressure reactor and used as a catalyst for the next catalytic reaction. After the catalyst was repeatedly used for 3 times, the selectivity of a synthesized product was 97.5%, and the yield was 96%.

### Example 15

1 mol cyclohexene oxide and 2.5 mmol [2-(hydroxymethyl)-3-(tributylphosphonium)-2-[(tributylphosphonium)methyl]propyl]tributylphosphonium triiodide were sequentially added into a 25 mL stainless steel high-pressure reactor with a polytetrafluoroethylene lining, the reactor was sealed, carbon dioxide at a proper pressure was charged, the reactor was slowly heated to 120° C., then the pressure of the carbon dioxide was controlled to be 10 MPa, and the reaction was performed for 0.1 h. The reactor was cooled to room temperature and depressurized, the carbon dioxide was absorbed by a saturated sodium bicarbonate solution, and the obtained liquid was distilled under reduced pressure to obtain a product. The peak time of the product was consistent with that of a standard sample through gas chromatographic analysis, indicating that the product was ethylene carbonate, the selectivity of the product was 99%, and the yield was 99%.

The distillation residue was transferred to the high-pressure reactor and used as a catalyst for the next catalytic reaction. After the catalyst was repeatedly used for 3 times, the selectivity of a synthesized product was 99%, and the yield was 98.5%.

### Comparative Example 1

Comparative Example 1 comprises most of the operating steps in Example 1, with the only difference being that the catalyst is selected from hydroxypropyltrimethylphosphonium bromide

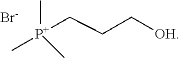

The selectivity of the obtained product was 98%, and the yield was 97%.

The distillation residue was transferred to the high-pressure reactor and used as a catalyst for the next catalytic reaction. After the catalyst was repeatedly used for 3 times, the selectivity of a synthesized product was 90%, and the yield was 85%.

### Comparative Example 2

Comparative Example 2 comprises most of the operating steps in Example 1, with the only difference being that the catalyst is selected from propyltrimethylphosphonium bromide

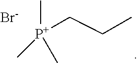

The selectivity of the obtained product was 90%, and the yield was 88%.

The distillation residue was transferred to the high-pressure reactor and used as a catalyst for the next catalytic reaction. After the catalyst was repeatedly used for 3 times, the selectivity of a synthesized product was 80%, and the yield was 73%.

As can be seen from the test results of Examples 1-15 and Comparative Examples 1-2, the hydroxy quaternary phosphonium salt catalyst containing a specific substituent can catalyze the reaction of carbon dioxide and an epoxy compound under mild conditions to obtain a cyclic carbonate, and the prepared cyclic carbonate has higher selectivity and yield; meanwhile, the catalyst has a longer catalytic life, and the synthesized cyclic carbonate still has relatively high selectivity and yield after being repeatedly used for 3 times.

In conclusion, the present disclosure can realize the synthesis of a cyclic carbonate from carbon dioxide and an epoxy compound through a cycloaddition reaction under mild reaction conditions by taking a brand-new hydroxy quaternary phosphonium salt as a catalyst, and the yield of the obtained cyclic carbonate is high and the catalytic effect is significant. Meanwhile, compared with the other existing catalysts in the comparative examples, the catalyst of the present disclosure has a longer catalytic life and better catalytic stability.

The present disclosure is further described above in conjunction with specific examples, but it should be understood that the specific description herein should not be construed as limiting the spirit and scope of the present disclosure, and that various modifications to the above examples made by those of ordinary skill in the art upon reading the specification fall within the protection scope of the present disclosure.

1. A catalyst for synthesizing a cyclic carbonate, wherein the catalyst is selected from compounds represented by a structural formula described below: 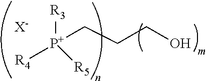
wherein R₃, R₄, and R₅ are each independently selected from a hydrogen atom and C₁-C₁₆ alkyl; X is a halogen atom; when n=1, m=2 or 3; when n=2, m=2; when n=3, m=1.

2. The catalyst for synthesizing a cyclic carbonate according to claim 1, wherein R₃, R₄, and R₅ are each independently selected from C₁-C₄ alkyl.

3. The catalyst for synthesizing a cyclic carbonate according to claim 1, wherein X is selected from one of Cl, Br, and I.

4. The catalyst for synthesizing a cyclic carbonate according to claim 1, wherein the catalyst is selected from one of compounds represented by structural formulas described below:

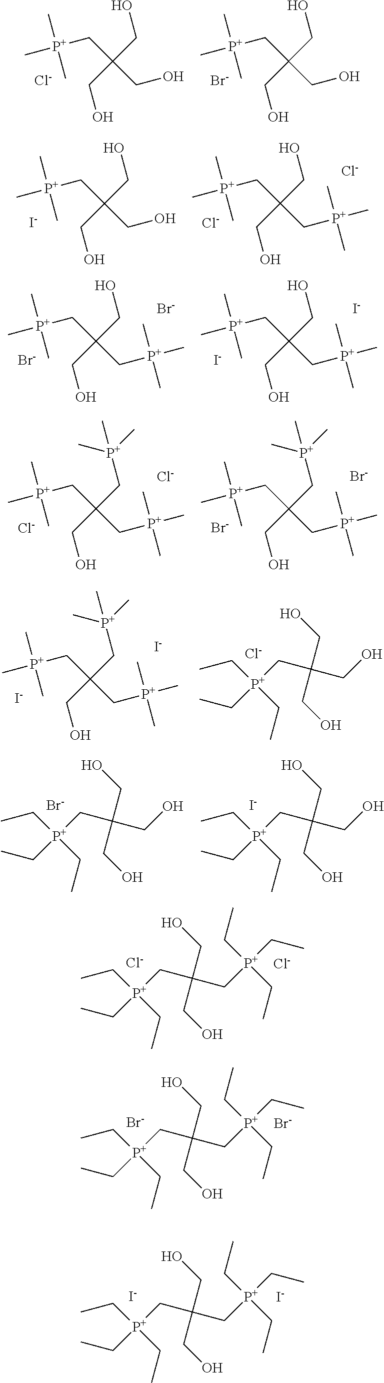

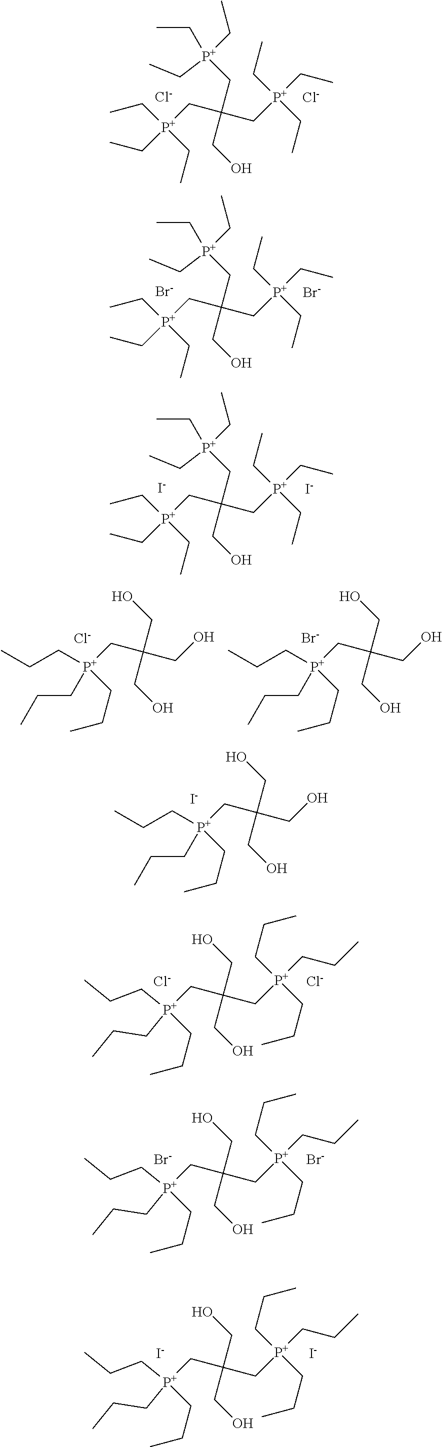

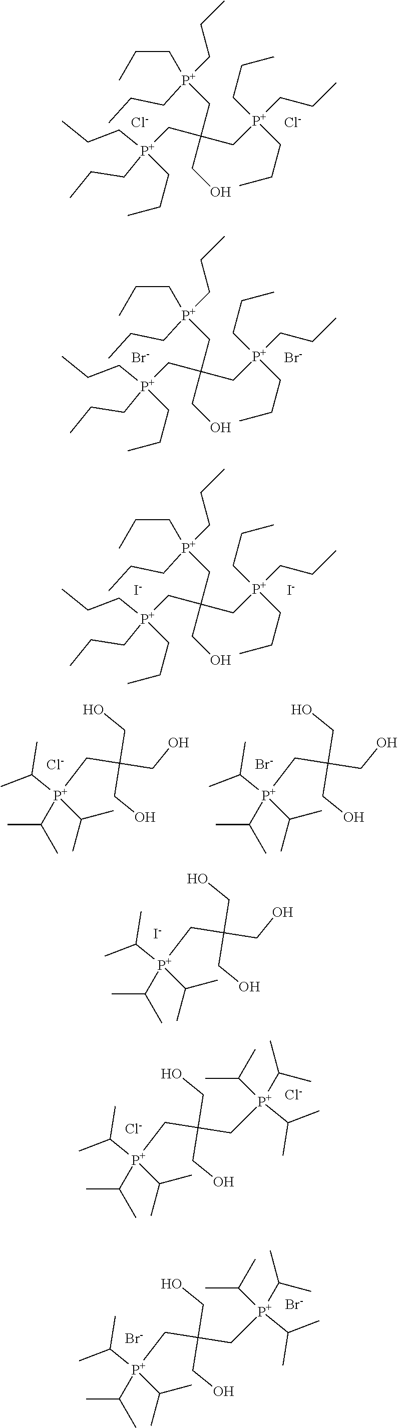

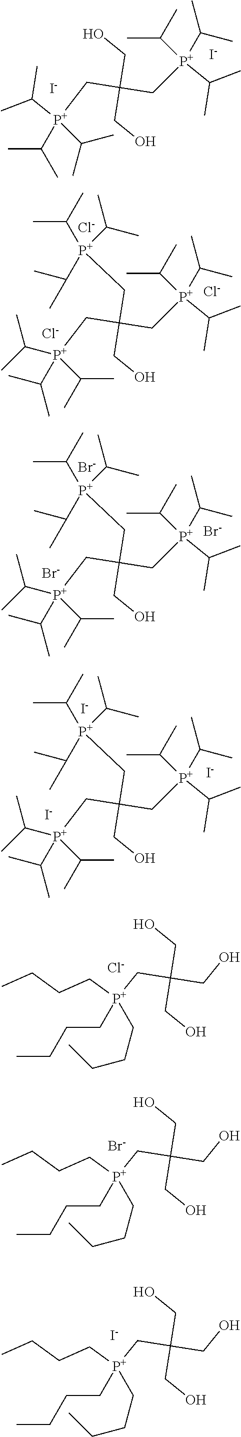

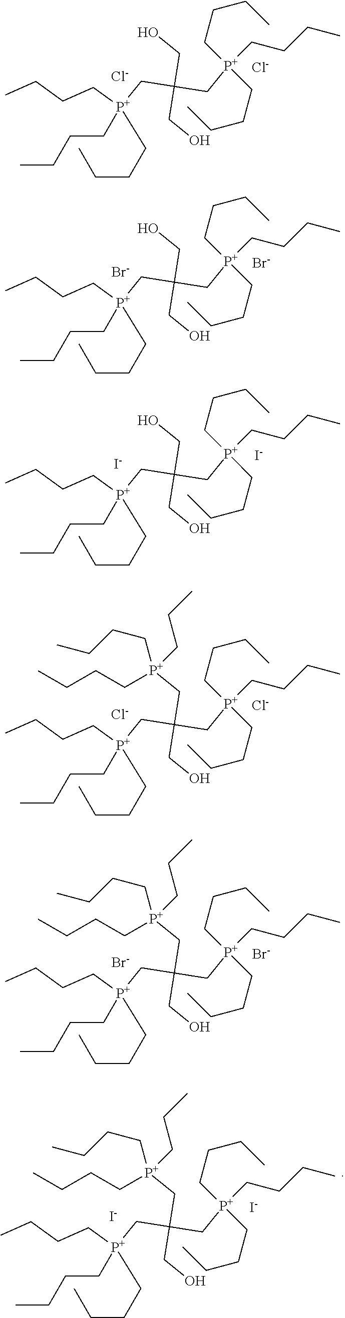

5. A synthesis method for a cyclic carbonate, wherein carbon dioxide and an epoxy compound are taken as raw materials and reacted under the catalysis of the catalyst according to claim 1 to synthesize the cyclic carbonate.

6. The synthesis method for a cyclic carbonate according to claim 5, wherein a structural formula of the epoxy compound is: 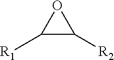
wherein when R₁=H, R₂ is one of H, CH₃, C₂H₅, CH₂Cl, C₂H₃, C₄H₉O, C₄H₉, C₆H₅, and C₇H₇O; when R₁≠H, the epoxy compound is cyclohexene oxide.

7. The synthesis method for a cyclic carbonate according to claim 5, wherein a molar ratio of the catalyst to the epoxy compound is 1×10⁻³-2.5×10⁻³:1.

8. The synthesis method for a cyclic carbonate according to claim 5, wherein a pressure of the reaction is 0.1-10 MPa.

9. The synthesis method for a cyclic carbonate according to claim 5, wherein a temperature of the reaction is 40-220° C.

10. The synthesis method for a cyclic carbonate according to claim 5, wherein a time of the reaction is 0.5-6 h.
